(19) United States
(12) Patent Application Publication
(10) Pub. No.: US 2025/0352338 A1
(43) Pub. Date: Nov. 20, 2025

(54) PROSTHETIC MEDICAL DEVICE DELIVERY APPARATUS

(71) Applicant: EDWARDS LIFESCIENCES CORPORATION, Irvine, CA (US)

(72) Inventor: Natanel Simcha Sirote, Zikhron Ya’akov (IL)

(21) Appl. No.: 19/281,887

(22) Filed: Jul. 28, 2025

Related U.S. Application Data
(63) Continuation of application No. PCT/US2024/013279, filed on Jan. 29, 2024.
(60) Provisional application No. 63/482,217, filed on Jan. 30, 2023.

(51) Int. Cl. A61F 2/24

(52) U.S. Cl. CPC A61F 2/2433

(57) ABSTRACT

A delivery apparatus can comprise a shaft comprising a proximal end portion coupled to a handle, a distal end portion configured to couple to a prosthetic medical device, and a central longitudinal axis extending therebetween. The delivery apparatus can further comprise a rotation gauge configured to provide a real-time indication of a rotational movement of the prosthetic medical device about the central longitudinal axis based on a rotational movement of the proximal end portion of the shaft about the central longitudinal axis and a transmission ratio of the shaft. The transmission ratio can be a ratio of a rotational movement of the distal end portion of the shaft to a rotational movement of the proximal end portion of the shaft, wherein the rotational movement of the proximal end portion of the shaft results in the rotational movement of the distal end portion of the shaft.

### CROSS REFERENCE TO RELATED APPLICATION

This application is a continuation of PCT Patent Application No. PCT/US2024/013279 filed on Jan. 29, 2024, which claims the benefit of U.S. Provisional Application No. 63/482,217, filed Jan. 30, 2023, each of these applications being incorporated by reference herein in its entirety.

### FIELD

The present disclosure relates to delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (such as stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (such as through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

The distal end of the delivery apparatus can flex, bend, twist, turn, and/or otherwise articulate to advance the prosthetic medical device (such as the prosthetic heart valve) past various turns, corners, constrictions, and/or other obstacles in the patient's vasculature. The delivery apparatus can comprise a handle at the proximal end of the delivery apparatus. A user (such as a clinician) can manipulate the handle during an implantation procedure to articulate the distal end of the delivery apparatus during the implantation procedure.

### SUMMARY

The foregoing and other objects, features, and advantages of the invention will become more apparent from the following detailed description, which proceeds with reference to the accompanying figures.

Described herein are prosthetic heart valves, delivery apparatuses, delivery apparatus stabilizers, and methods for implanting prosthetic heart valves. The disclosed delivery apparatuses and methods can, for example, can provide for improved positioning of a prosthetic medical device during an implantation procedure. For example, a delivery apparatus can comprise a handle and a rotation gauge that provides a real-time indication of the prosthetic medical device's rotational movement during the implantation procedure. The real-time indication of the prosthetic medical device's rotational movement can help a user (such as a clinician) better estimate the prosthetic medical device's position within the patient's vasculature. As such, the devices and methods disclosed herein can, among other things, overcome one or more of the deficiencies of typical prosthetic heart valves and their delivery apparatuses.

A delivery apparatus for implanting a prosthetic medical device can comprise a handle and at least one shaft coupled to the handle.

In some examples, the shaft can comprise a central longitudinal axis along which the shaft can be configured to extend.

In some examples, the shaft can comprise a distal end portion configured to be coupled to the prosthetic medical device.

In some examples, the distal end portion of the shaft can be configured to be coupled to a valve mounting portion.

In some examples, the delivery apparatus can comprise a rotation gauge configured to provide an indication of a rotational movement about the central longitudinal axis of the prosthetic medical device coupled to the distal end portion of the shaft.

In some examples, the indication of the rotational movement of the prosthetic medical device can be based on a rotational movement of the proximal end portion of the shaft about the central longitudinal axis and a transmission ratio.

In some examples, the rotational movement of the prosthetic medical device can be equal to the rotational movement of the proximal end portion of the shaft multiplied by the transmission ratio.

In some examples, the transmission ratio can comprise a ratio of a rotational movement of the distal end portion of the shaft about the central longitudinal axis and a rotational movement of the proximal end portion of the shaft about the central longitudinal axis.

In some examples, the rotational movement of the proximal end portion of the shaft can result in the rotational movement of the distal end portion of the shaft.

In some examples, the rotation gauge can comprise a gyroscope coupled to the handle.

In some examples, the rotation gauge can comprise a rotational sensor coupled to the shaft.

In some examples, the rotation gauge can comprise a gear train comprising a gear fixedly coupled to the shaft and a ring gear disposed around the handle.

In some examples, the gear train can have a compound gear ratio, wherein the compound gear ratio can be equal to the transmission ratio.

In some examples, the rotation gauge can comprise an annular vial and a spirit bubble disposed within the annular vial.

In some examples, the rotation gauge can comprise a channel disposed along a circumference of the handle and a ball bearing disposed within the channel.

In some examples, a delivery apparatus can comprise: a shaft, a handle, and a rotation gauge. The shaft can comprise a proximal end portion, a distal end portion, and a central longitudinal axis extending from the proximal end portion to the distal end portion, wherein the distal end portion of the shaft is configured to be coupled to a prosthetic medical device. The handle can be coupled to the proximal end portion of the shaft. The rotation gauge can be coupled to the proximal end portion of the shaft. The rotation gauge can be configured to provide a real-time indication of a rotational movement of the prosthetic medical device about the central longitudinal axis based on a rotational movement of the proximal end portion of the shaft and a transmission ratio of the shaft.

In some examples, a delivery apparatus can comprise a shaft extending along a central longitudinal axis. The shaft can comprise a proximal end portion and a distal end portion. The delivery apparatus can further comprise a handle coupled to the proximal end portion of the shaft and a rotation gauge coupled to the handle. The rotation gauge can be configured to provide an indication of a rotational movement of the distal end portion of the shaft about the central longitudinal axis. The indication can be based on a rotational movement of the proximal end portion of the shaft about to the central longitudinal axis and a transmission ratio between the distal end portion of the shaft and the proximal end portion of the shaft.

In some examples, a delivery apparatus for implanting a prosthetic medical device can comprise a shaft extending along a central longitudinal axis. The shaft can comprise a proximal end portion and a distal end portion. The delivery apparatus can further comprise
a valve mounting portion coupled to the distal end portion of the shaft, wherein the valve mounting portion can be configured to receive the prosthetic medical device. The delivery apparatus can further comprise a handle coupled to the proximal end portion of the shaft and a rotation gauge disposed on the handle. The rotation gauge can be configured to provide an indication of a degree of rotational movement of the valve mounting portion about the central longitudinal axis based on a degree of rotational movement of the handle about the central longitudinal axis and a transmission ratio between the distal end portion of the shaft and the handle.

In some examples, a method of providing an indication of a rotational movement of a prosthetic heart valve coupled to a distal end portion of a delivery apparatus can comprise a step of measuring an angular position of a proximal end portion of the delivery apparatus relative to a central longitudinal axis of the delivery apparatus. The method can further comprise a step of determining an angular position of the prosthetic heart valve relative to the central longitudinal axis by multiplying the angular position of the proximal end portion of the delivery apparatus by a transmission ratio. The transmission ratio can be a ratio of a rotational movement of the distal end portion of the delivery apparatus to a rotational movement of the proximal end portion of the delivery apparatus. The method can further comprise a step of displaying the angular position of the prosthetic heart valve.

In some examples, an assembly can comprise one or more of the components recited in Examples 1-21 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The above method(s) can be performed on a living animal or on a simulation, such as on a cadaver, cadaver heart, anthropomorphic ghost, simulator (e.g., with body parts, heart, tissue, etc. being simulated).

### BRIEF DESCRIPTION OF THE DRAWINGS

FIG. 1 is a perspective view of a prosthetic heart valve, according to one example.

FIG. 2 is a side view of a prosthetic heart valve delivery apparatus, according to one example.

FIG. 3 is an exploded side view of a prosthetic heart valve delivery apparatus with an electronic rotation gauge, according to an example.

FIG. 4 is a side view of a prosthetic heart valve delivery apparatus with an electronic rotation gauge, according to an example.

FIGS. 5A and 5B are axial cross-sectional views of exemplary prosthetic heart valve delivery apparatuses of FIG. 4, including a rotary encoder and a rotary potentiometer, respectively.

FIG. 6 is a side view of a prosthetic heart valve delivery apparatus with a mechanical rotation gauge, according to an example.

FIG. 7 is an axial cross-sectional view of the prosthetic heart valve delivery apparatus of FIG. 6.

FIG. 8 is a side view of a prosthetic heart valve delivery apparatus with a spirit level rotation gauge, according to an example.

FIG. 9 is an axial cross-sectional view of the prosthetic heart valve delivery apparatus of FIG. 8.

FIG. 10 is a side view of a prosthetic heart valve delivery apparatus with a ball bearing rotation gauge, according to an example.

FIG. 11 is an axial cross-sectional view of the prosthetic heart valve delivery apparatus of FIG. 10.

FIG. 12 is an illustration of an example computing environment in which some example rotation gauges can be implemented, according to an example.

FIG. 13 is a side view of a prosthetic heart valve, according to an example.

FIG. 14 is a side view of a prosthetic heart valve, according to an example.

FIG. 15A is a perspective view of a prosthetic heart valve, according to one example.

FIG. 15B is a perspective view of the prosthetic valve of FIG. 15A with the components on the outside of the frame shown in transparent lines for illustrative purposes.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used in this application and in the claims, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (such as out of the patient's body), while distal motion of the device is motion of the device away from the user and toward the implantation site (such as into the patient's body). The terms “longitudinal” and “axial” refer to an axis extending in the proximal and distal directions, unless otherwise expressly defined.

As used in this application and in the claims, the term “radial” refers to an axis perpendicular to the longitudinal axis.

As used herein, “e.g.” means “for example,” and “i.e.” means “that is.”

### INTRODUCTION TO THE DISCLOSED TECHNOLOGY

Described herein are examples of a steerable delivery apparatus (sometimes referred to as a steerable catheter) that can be used to navigate a subject's vasculature to deliver an implantable, expandable medical device (e.g., a prosthetic heart valve), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems are described herein that, in some examples, can provide a user of the delivery apparatus with a real-time indication of a rotational movement or angular position of the prosthetic medical device about a central longitudinal axis of the delivery apparatus to better improve the positioning of the prosthetic medical device during an implantation procedure.

The delivery apparatus can comprise a shaft extending along the central longitudinal axis. The shaft can comprise a proximal end portion and a distal end portion. The proximal end portion of the shaft can be coupled to a handle, which in some examples can also be aligned with the central longitudinal axis. The prosthetic medical device can be coupled to the distal end portion of the shaft.

During the implantation procedure, the prosthetic medical device may be “clocked” or rotated about the central longitudinal axis to align the prosthetic medical device with a patient's vasculature. The prosthetic medical device can be rotated by manipulating a proximal portion of the delivery apparatus. In some examples, the prosthetic medical device can be clocked by rotating the handle about the central longitudinal axis. Since the handle is coupled to the prosthetic medical device via the shaft, a rotational movement of the handle can result in a corresponding rotational movement of the prosthetic medical device. In some examples, the handle can comprise a rotatable knob operably coupled to the proximal end portion of the shaft. In such examples, the prosthetic medical device can be clocked by turning the knob, which can result in the shaft and the prosthetic heart valve rotating about the central longitudinal axis.

During the implantation procedure, a user (such as a clinician) of the delivery apparatus can estimate the rotational movement of the prosthetic medical device based on the rotational movement of the proximal portion of the delivery apparatus (such as the handle or the rotatable knob). However, the accuracy of this estimate can be further improved by accounting for torsional deformation of the shaft. For example, a torque applied to the shaft by the rotating handle can cause the shaft to torsionally deform. When the shaft torsionally deforms, the degree of rotational movement of the distal end portion of the delivery apparatus (for example, the distal end portion of the shaft to which the prosthetic heart valve is coupled) may differ from the degree of rotational movement of the proximal end portion of the delivery apparatus (for example, the proximal end portion of the shaft) by a “transmission ratio.”

The inventors have discovered that it can be desirable include a rotation gauge on the delivery apparatus that accounts for the transmission ratio. The rotation gauge can provide the user with a more accurate indication of the rotational movement or angular position of the prosthetic medical device coupled to the distal end portion of the delivery apparatus. These more accurate indications can make the delivery apparatus easier to use and can beneficially lead to more favorable surgical outcomes.

### EXAMPLES OF THE DISCLOSED TECHNOLOGY

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

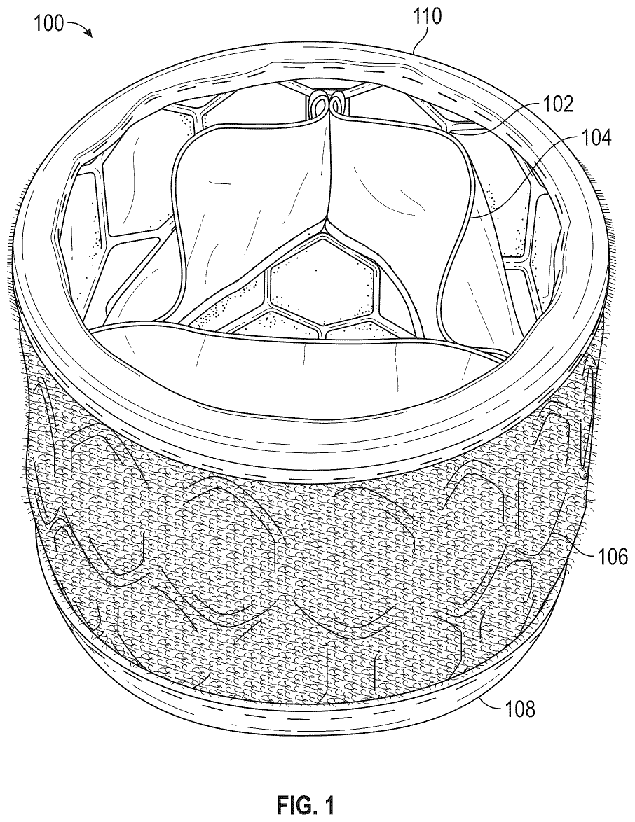

As shown in FIG. 1, the prosthetic valve 100 can include a frame 102 and a plurality of leaflets 104 can be situated at least partially within the frame 102. The prosthetic valve 100 can also include an outer covering 106 situated about the frame 102. As shown in FIG. 1, the prosthetic valve 100 includes an inflow end 108 and an outflow end 110. The terms “inflow” and “outflow” are related to the normal direction of blood flow (e.g., antegrade blood flow) through the prosthetic valve 100. For example, the leaflets 104 can allow blood flow through the valve 100 in a direction from the inflow end 108 to the outflow end 110 and prevent the reverse flow (e.g., prevent flow in a direction from the outflow end 110 to the inflow end 108).

The frame 102 can be made of any of various suitable plastically-expandable materials (e.g., stainless steel, etc.) or self-expanding materials (e.g., Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (e.g., the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The outer covering 106 can be wholly or partly formed of any suitable biological material, synthetic material (for example, any of various polymers), or combinations thereof. In some examples, the outer covering 106 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer covering 106 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the outer covering 106 can comprise a sponge material or foam, such as polyurethane foam. In some examples, the outer covering 106 can comprise natural tissue, such as pericardium (for example, bovine pericardium, porcine pericardium, equine pericardium, or pericardium from other sources).

Further details of the prosthetic heart valve and its variants are described in U.S. Pat. No. 11,185,406, which is incorporated by reference herein in its entirety.

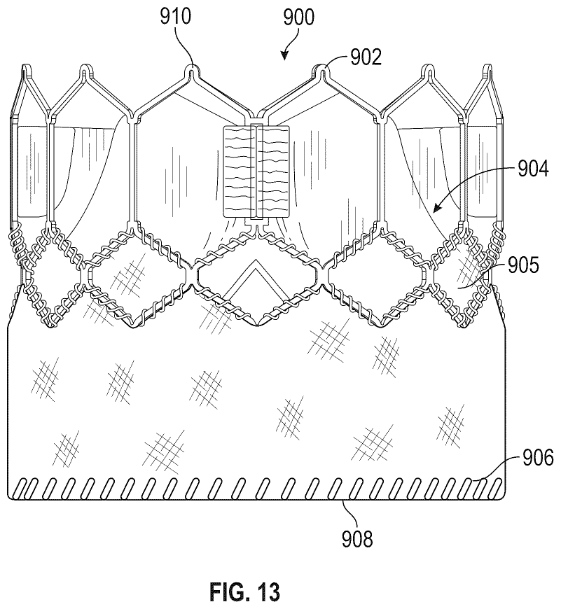

FIG. 13 shows a prosthetic heart valve 900, according to an example. The prosthetic heart valve 900 can comprise a frame 902, a valvular structure 904, an inner skirt 905 (which is also referred to herein as an “inner sealing member”), an outer skirt 906, an inflow end portion 908, and an outflow end portion 910. The inner skirt 905 can be coupled to an inner surface of the frame 902.

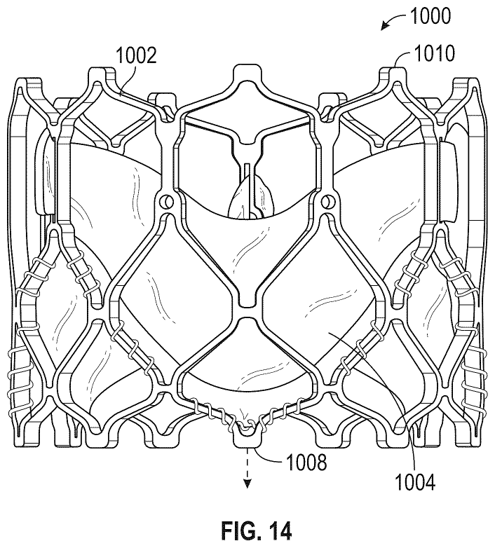

FIG. 14 shows a prosthetic heart valve 1000, according to an example. The prosthetic heart valve 1000 can comprise a frame 1002, a valvular structure 1004, an inflow end portion 1008, and an outflow end portion 1010. One exemplary difference between the prosthetic heart valve 1000 and prosthetic heart valves 100, 900 described throughout this application is that the valvular structure 1004 can be coupled directly to the frame 1002 instead of to an inner skirt coupled to the frame 1002.

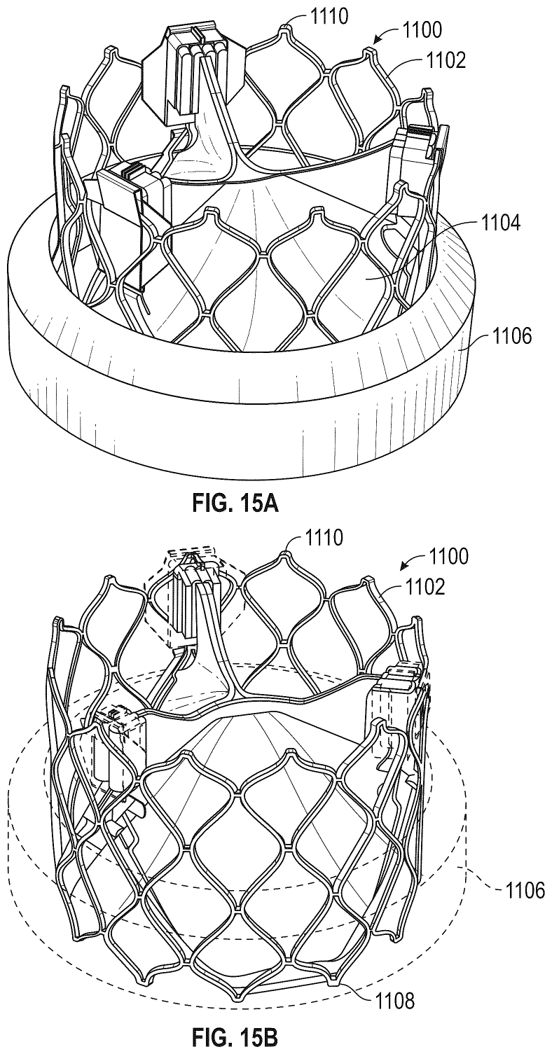

FIGS. 15A and 15B show a prosthetic heart valve 1100, according to an example. The prosthetic heart valve 1100 can comprise a frame 1102, a valvular structure 1104, an outer skirt 1106, an inflow end portion 1108, and an outflow end portion 1110.

### Delivery Apparatus

Described herein are examples of a steerable delivery apparatus 200 (sometimes referred to as a “steerable catheter” and/or a “balloon catheter”) that can be used to navigate a subject's vasculature to deliver an implantable, expandable medical device (for example, a prosthetic heart valve), tools, agents, or other therapy to a location within the body of a subject. Examples of procedures in which the steerable catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

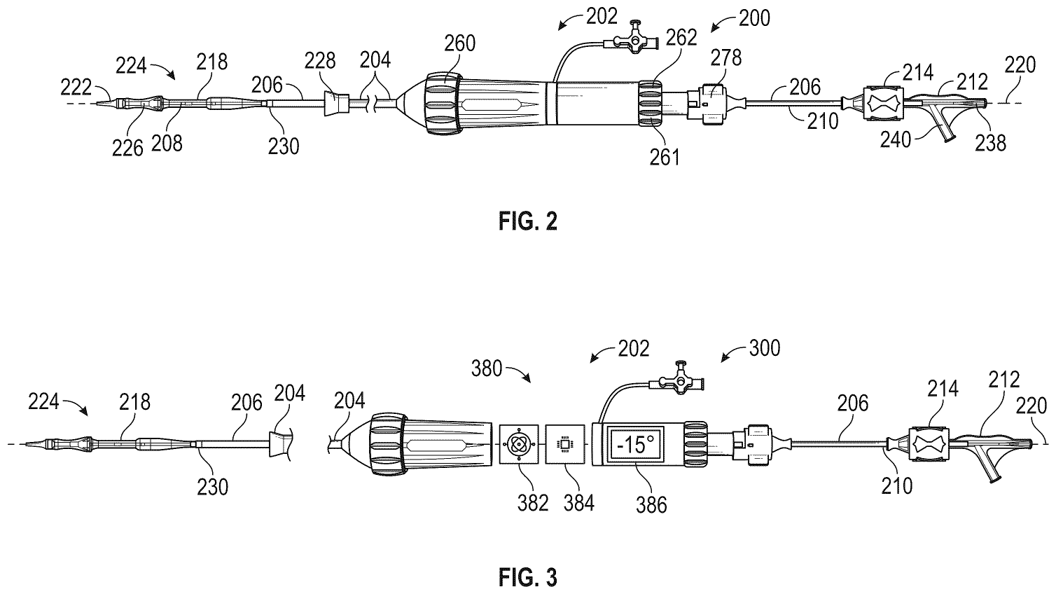

FIG. 2 shows the delivery apparatus 200, according to one example, that can be used to implant a prosthetic medical device. In some examples, the delivery apparatus 200 can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic heart valve into a heart.

The delivery apparatus 200 in the illustrated example of FIG. 2 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic heart valve at an implantation site in a patient's body.

The intermediate shaft 206 can include a proximal end portion 210 that extends proximally from a proximal end of the handle 202, to an adaptor 212. In some examples, a rotatable knob 214 can be mounted on the proximal end portion 210 and can be configured to rotate the intermediate shaft 206 around the central longitudinal axis 220 and relative to the outer shaft 204. In some examples, the knob 214 can be directly coupled to the intermediate shaft 206. In some examples, the knob 214 can be directly coupled to and/or arranged around a portion of an adaptor 212 coupled to the intermediate shaft 206. In some examples, the knob 214 can be spaced axially away from the adaptor 212.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion 230 that extends distally beyond a distal end of the outer shaft 204 when a distal end of the outer shaft 204 is positioned away from an inflatable catheter balloon 218 (also referred to herein as a “balloon”) of the delivery apparatus 200. In some examples, a distal end portion of the inner shaft 208 can extend distally beyond the distal end portion 230 of the intermediate shaft 206. In some examples, the catheter balloon 218 can be coupled to the distal end portion 230 of the intermediate shaft 206.

In some examples, a distal end of the catheter balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the catheter balloon 218 can overlay a valve mounting portion 224 of a distal end of the delivery apparatus 200 and a distal end portion of the catheter balloon 218 can overly a distal shoulder 226 of the delivery apparatus 200. The valve mounting portion 224 and the intermediate portion of the catheter balloon 218 can be configured to receive a prosthetic heart valve in a radially compressed state. For example, as shown schematically in FIG. 2, a prosthetic heart valve (for example, prosthetic heart valve 100) can be mounted around the catheter balloon 218, at the valve mounting portion 224 of the delivery apparatus 200.

The balloon shoulder assembly, including the distal shoulder 226, is configured to maintain the prosthetic heart valve 100 (or other prosthetic medical device) at a fixed position on the catheter balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic heart valve 100 is mounted in the radially compressed state on the valve mounting portion 224 (as shown in FIG. 2) and during delivery of the prosthetic heart valve to the target implantation site. As such, the distal tip portion 228 can be configured to resist movement of the prosthetic heart valve 100 relative to the catheter balloon 218 proximally, in the axial direction, relative to the catheter balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the catheter balloon 218. As such, fluid from the fluid source can flow to the fluid passageway from the annular space to inflate the catheter balloon 218 and radially expand and deploy the prosthetic heart valve 100.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Publication No. WO2022/046585, which is incorporated by reference herein.

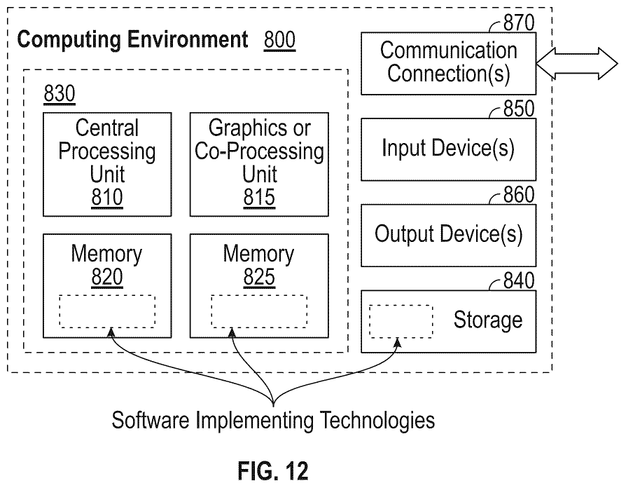

FIG. 3 illustrates an exploded side view of a delivery apparatus 300 with an electronic rotation gauge 380 (which is also referred to herein as an “electronic gauge assembly” or a “digital rotation gauge”), according to an example. The electronic rotation gauge 380 can be configured to provide a real-time indication of a rotational movement about the central longitudinal axis 220 of a prosthetic heart valve (for example, the prosthetic heart valve 100) coupled to the valve mounting portion 224 of the delivery apparatus 300. As illustrated in FIG. 3, the electronic rotation gauge 380 can comprise an electronic assembly configured to operate as a computing system (for example, the computing system illustrated in FIG. 12). Thus, the electronic rotation gauge 380 can include various electronic components forming an electronic circuit, such as a controller, a memory unit, a communication unit, a battery, a storage device, an input/output device, etc. For example, the electronic rotation gauge 380 can include an input device comprising an inertial measurement unit (IMU) 382 configured to measure a rotational movement (for example, a position, a displacement, a velocity, or an acceleration) of a proximal end portion of the delivery apparatus 300 about the central longitudinal axis 220. The electronic rotation gauge 380 can further include a controller 384 configured to determine the rotational movement of the prosthetic heart valve about the central longitudinal axis 220 based on the rotational movement measured by the IMU 382 and a transmission ratio of the delivery apparatus 300. The electronic rotation gauge 380 can further include an output device comprising an electronic display 386 configured to display an indication of the rotational movement of the prosthetic heart valve about the central longitudinal axis 220 to a user (such as a clinician) operating the delivery apparatus 300.

As illustrated in FIG. 3, at least a portion of the electronic rotation gauge 380 (such as the IMU 382 and/or the controller 384) can be disposed within the handle 202 of the delivery apparatus 300. In some examples, at least a portion of the electronic rotation gauge 380 (such as the electronic display 386) can be coupled to an outer surface of the handle 202. In some examples, the electronic rotation gauge 380 can be permanently attached to the handle 202. In some examples, a portion (such as a battery) or the entirety of the electronic rotation gauge 380 can be removably coupled to the handle 202.

The IMU 382 can be configured to measure the rotational movement of the proximal end portion of the delivery apparatus 300. As used herein, the “proximal end portion” of the delivery apparatus broadly refers to any portion of the delivery apparatus 300 distally disposed relative to the distal end portion 230 of the intermediate shaft 206, including but not limited to the handle 202, the proximal end portion 210 of the intermediate shaft 206, the knob 214, and the adaptor 212. The IMU 382 may be configured to measure the rotational movement of the proximal end portion of the delivery apparatus 300 as an angular position (which is also referred to herein as an “angular orientation”), an angular displacement (for example, a relative displacement relative to a reference orientation), an angular velocity, and/or an angular acceleration. The IMU 382 can comprise one or more rotational sensors (such as an accelerometer, a gyroscope, or a magnetometer) that measures a 3D orientation, a 3D velocity, a 3D gravitational force, or a 3D acceleration of the intermediate shaft 206. The IMU 382 can be electronically coupled to the controller, battery, and/or other electronic components. The IMU 382 can be configured to generate sensor data (such as a value or a series of values indicating a position, displacement, velocity, and/or acceleration of the proximal end of the delivery apparatus 300) and send the sensor data to the controller 384. The sensor data can be in any format (for example, digital, analog, etc.) readable by the controller 384.

The IMU 382 may be coupled to any of the handle 202, the intermediate shaft 206 (for example, the proximal end portion 210 of the intermediate shaft 206), the knob 214, the adaptor 212, or any other part of the proximal end portion of the delivery apparatus 300. As shown in the example illustrated in FIG. 3, where the rotational movement of the proximal end portion of the delivery apparatus 300 comprises a rotational movement of the handle 202 about the central longitudinal axis 220, the IMU 382 can be fixedly coupled to the handle 202 such that the IMU 382 synchronously rotates about the central longitudinal axis 220 with the handle 202. In some examples where the rotational movement comprises a rotational movement of the proximal end portion 210 of the intermediate shaft 206 about the central longitudinal axis 220, the IMU 382 can be fixedly coupled to a portion of the intermediate shaft 206 (for example, the proximal end portion 210 or a portion of the intermediate shaft 206 disposed within the handle 202) such that the IMU 382 synchronously rotates about the central longitudinal axis 220 with the proximal end portion 210 of the intermediate shaft 206. In some examples where the rotational movement of the proximal end portion of the delivery apparatus 300 comprises a rotation of the knob 214 coupled to the proximal end portion 210 of the intermediate shaft 206, the IMU 382 can be fixedly coupled to the knob 214 such that the IMU 382 synchronously rotates with the knob 214 relative to the handle 202. In any of these examples, the rotational movement may be a relative movement (for example, a rotational movement relative to a reference frame, position, or orientation of the handle 202) or an absolute movement.

In some examples, the electronic rotation gauge 380 can comprise a plurality of IMUs 382. For example, the electronic rotation gauge 380 can comprise a first IMU 382 coupled to the handle 202, wherein the first IMU 382 can be configured to measure the absolute rotational movement of the handle 202 about the central longitudinal axis 220. The electronic rotation gauge 380 can comprise a second IMU 382 coupled to the intermediate shaft 206 or the knob 214, wherein the second IMU 382 can be configured to measure a rotational movement of the intermediate shaft 206 about the central longitudinal axis 220 and relative to the handle 202. By accounting for the rotational movements of both the handle 202 and the intermediate shaft 206, the electronic rotation gauge 380 can more accurately determine an absolute rotational movement of the prosthetic heart valve.

The controller 384 can be configured to manage and control various aspects of the operation of the electronic circuit, including battery management, on/off status of input devices (such as the IMU 382) and/or output devices (such as the electronic display 386), control of power levels, sensor measurement, analysis of the measured sensor data, control of output devices, communication with external computing devices, etc. For example, the controller 384 can be configured to receive sensor data from the IMU 382, wherein the sensor data comprises a value or a series of values indicating an angular position, an angular displacement, an angular velocity, or an angular acceleration of the proximal end portion of the delivery apparatus 300 about the central longitudinal axis 220. The controller 384 can subsequently determine the rotational movement of the prosthetic heart valve about the central longitudinal axis 220—wherein the prosthetic heart valve is coupled to the distal end portion 230 of the shaft 206 at the distal end portion of the delivery apparatus 300—based on the sensor data received from the IMU 382 and the transmission ratio between the proximal end portion and the distal portion of the delivery apparatus 300. As used herein, the term “distal end portion” of the delivery apparatus refers to any portion of the delivery apparatus 300 distally disposed relative to the handle 202, including but not limited to the distal end portion 230 of the intermediate shaft 206, the catheter balloon 218, and the valve mounting portion 224. The controller 384 can then send a signal to the electronic display 386 to display an indication of the rotational movement of the prosthetic heart valve.

The transmission ratio is a ratio of the rotational movement of the distal end portion of the delivery apparatus 300 (to which the prosthetic heart valve is coupled) to the rotational movement of the proximal end portion of the delivery apparatus 300 which results in the rotational movement of the distal end portion and the prosthetic heart valve coupled to the distal end portion. In some examples, the transmission ratio can more specifically be the ratio of the rotational movement of the distal end portion 230 of the intermediate shaft 206 about the central longitudinal axis 220 to the rotational movement about the central longitudinal axis 220 of a portion of the intermediate shaft 206 disposed within the handle 202, wherein the rotational movement of the portion of the intermediate shaft 206 results in the movement of the distal end portion 230 of the intermediate shaft 206. In some examples, the transmission ratio can more specifically be the ratio of the rotational movement the distal end portion 230 of the intermediate shaft 206 to the rotational movement of the proximal end portion 210 of the intermediate shaft 206, wherein both rotational movements are about the central longitudinal axis 220 and wherein the rotational movement of the proximal end portion 210 results in the rotational movement of the distal end portion 230. In some examples, the transmission ratio can more specifically be the ratio of the rotational movement of the distal end portion 230 of the intermediate shaft 206 and the rotational movement of the knob 214 (which can be coupled to the proximal end portion 210 of the intermediate shaft 206), wherein the rotational movement of the knob 214 results in the rotational movement of the distal end portion 230 of the intermediate shaft 206. In some examples, the transmission ratio can be less than one, signifying that a degree, magnitude, or extent of the rotation of the proximal end portion of the delivery apparatus 300 about the central longitudinal axis 220 is greater than a resulting degree, magnitude, or extent of the rotation of the distal end portion of the delivery apparatus 300. However, some examples of the transmission ratio can be equal to or greater than one, signifying that the degree, magnitude, or extent of the rotation of the proximal end portion of the delivery apparatus 300 about the central longitudinal axis 220 is equal to or less than than the resulting degree, magnitude, or extent of the rotation of the distal end portion of the delivery apparatus 300, respectively.

The controller 384 may use any suitable algorithm to determine the rotational movement of the prosthetic heart valve about the central longitudinal axis 220 based on the sensor data from the IMU 382 and the transmission ratio. In some examples where the IMU 382 is configured to measure an angular acceleration of the proximal end portion of the delivery apparatus 300, the controller 384 can be configured to determine an angular position (which is also referred to herein as an “angular orientation”) of the prosthetic heart valve by first multiplying the angular acceleration of the proximal end portion of the delivery apparatus 300 about the central longitudinal axis 220 (for example, the angular acceleration of the handle 202, the proximal end portion 210 of the intermediate shaft 206, or the knob 214 about the central longitudinal axis 220) by the transmission ratio to determine the angular acceleration of the distal end portion of the delivery apparatus 300 (for example, the distal end portion 230 of the intermediate shaft 206 and/or the valve mounting portion 224 coupled to the distal end portion 230) about the central longitudinal axis 220, wherein the distal end portion is coupled to the prosthetic heart valve. The controller 384 can subsequently double-integrate the angular acceleration of the distal end portion of the delivery apparatus 300 to determine the angular position indicative of the rotational movement of the prosthetic heart valve about the central longitudinal axis 220.

In some examples, the controller 384 can be further configured to determine the transmission ratio prior to determining the angular position of the prosthetic heart valve. In some examples, the transmission ratio may be a constant value. Thus, in such examples, the transmission ratio may be a predetermined value retrieved by the controller 384 from a memory unit of the electronic assembly. In some examples, the transmission ratio may be received as input from the user of the delivery apparatus 300. For example, the user can input a shaft identifier corresponding to the intermediate shaft 206 into the electronic assembly. The controller 384 can determine the transmission ratio corresponding to the shaft identifier. In some examples, the controller 384 can determine the transmission ratio based at least in part on a wireless communication received from an external computing device.

In some examples, the transmission ratio may vary based on one or more parameters, such as time, environmental conditions (such as temperature or humidity), a length of the intermediate shaft 206 (for example, a length between the proximal end portion 210 and the distal end portion 230), an angular position of the intermediate shaft 206, an amount of torsion being experienced by the intermediate shaft 206, or a deployment position of the intermediate shaft 206 and/or the delivery apparatus 300. In such examples, the controller 384 can determine the transmission ratio based on any combination of the one or more measured parameters, instructions stored in the memory unit of the electronic assembly, sensor data from various input devices electronically coupled to the controller 384, and/or wireless communications received from external computing devices.

In the illustrated example, the controller 384 may be disposed within the handle 202. In some examples, the controller 384 may be disposed the outer surface of the handle 202. In some examples, the controller 384 can be wirelessly connected to the IMU 382, thereby allowing the controller 384 to be physically separate from the handle 202. In such examples, the controller 384 can be part of an external computing device (such as a smartphone, a wearable device, a computer, or a server) in wireless communication with IMU 382 and/or the electronic display 386.

The electronic display 386 can be configured to show an indication of the rotational movement of the prosthetic heart valve to the user of the delivery apparatus 300. The electronic display 386 can comprise any one of a liquid crystal (LCD) display, a light-emitting diode (LED) display, an organic light emitting diode (OLED) display, an active-matrix organic light-emitting diode (AMOLED) display, an electroluminescent display, a plasma display, a thin-film transistor (TFT) display, an electrophoretic display, an electrowetting display, an electrochromic display, or any other suitable type of electronic display. The electronic display 386 can be configured to show any suitable indication of the rotational movement of the prosthetic heart valve. In some examples, the rotational movement of the prosthetic heart valve may be indicated by the angular position of the prosthetic heart valve. The angular position may be absolute or may be relative to the reference frame, position, or orientation of the proximal end portion of the delivery apparatus 300 (for example, a reference frame of the handle 202, a reference frame of the knob 214, or a reference frame of the proximal end portion 210 of the intermediate shaft 206). The electronic display 386 may indicate the angular position as a number, as a position on a dial, as a location on a graph or scale, as an image (for example, a mockup of the prosthetic heart valve and/or the delivery apparatus 300), or as any other suitable representation of angular position.

The electronic display 386 is illustrated as disposed on the outer surface of the handle 202. In some examples, the electronic display 386 can be configured to rotate about the central longitudinal axis 220 such that the electronic display 386 remains visible to the user as the handle 202 is manipulated during the implantation procedure. Although the electronic display 386 is illustrated as rectangular, some examples of the electronic display 386 can be any suitable shape (for example, circular). In some examples, the electronic display 386 can extend circumferentially around the outer surface of the handle 202 such that at least a portion of the electronic display 386 is always visible to the user. The electronic display 386 may be coupled to any suitable portion of the delivery apparatus 300 (such as on or near the proximal end portion 210 of the intermediate shaft 206, the knob 214, or the adaptor 212). In some examples, the electronic display 386 can be physically separate from the handle 202. In such examples, the electronic display 386 can be part of an external computing device (such as a smartphone, a wearable device, a monitor, or a computer) in wireless communication with IMU 382 and/or the controller 384.

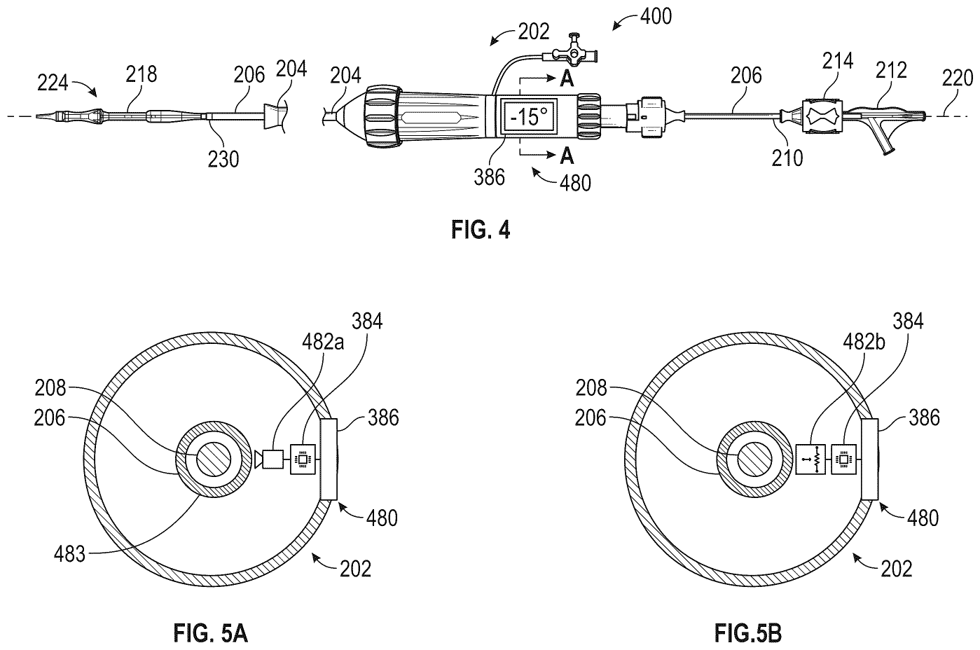

FIG. 4 illustrates a side view of a delivery apparatus 400 with an electronic rotation gauge 480, according to an example. The electronic rotation gauge 480 can comprise a rotational sensor 482, the controller 384, and the electronic display 386. One exemplary difference between the electronic rotation gauge 480 and the electronic rotation gauge 380 illustrated in FIG. 3 is that some examples of the rotational sensor 482 can comprise a rotary encoder (FIG. 5A) or a rotary potentiometer (FIG. 5B) coupled to the intermediate shaft 206 instead of the IMU 382 coupled to the handle 202. However, it should be understood that the rotational sensor 482 may be any suitable type of sensor configured to measure angular positions.

FIG. 5A shows an axial cross-section of the handle 202 about axis A-A, according to one example. Although the illustrated example of the rotary encoder 482a is disposed at or near axis A-A, the rotary encoder 482a can be disposed anywhere along the axial length of the handle 202 and/or the intermediate shaft 206 (for example, at or near the proximal end portion 210 of the intermediate shaft 206 or at or near the knob 214). The cross-section shows the rotational sensor 482 embodied as the rotary encoder 482a (which is also referred to herein as a “shaft encoder”). The rotary encoder 482a can be configured to determine an angular position of the intermediate shaft 206 about the central longitudinal axis 220 and relative to the handle 202 based on a progression of the rotary encoder 482a along a scale 483 extending along the circumference of the intermediate shaft 206. Although the illustrated rotary encoder 482a is configured to measure the angular position of the portion of the intermediate shaft 206 disposed within the handle 202, various examples of the rotary encoder 482a can be positioned elsewhere on the delivery apparatus (for example, at or near the proximal end portion 210 of the intermediate shaft 206 or the knob 214) to measure other rotational movements. The rotary encoder 482a may be an absolute rotary encoder (configured to measure an absolute angular position) or may be an incremental rotary encoder (configured to measure changes in angular position). The rotary encoder 482a can be an optical encoder (as shown in FIG. 5A), a magnetic encoder, a capacitive encoder, an inductive encoder, or any other suitable type of encoder.

The scale 483 can comprise at least one graduated marking (such as an optical marking, a magnetic marking, etc.) disposed on the circumference of the intermediate shaft 206 for the rotary encoder 482a to read. However, since some examples of rotary encoders (such as optical encoders) do not require scales to operate, the scale 483 can be an optional component.

Although the rotary encoder 482a shown in FIG. 5A is configured to measure the angular position of the intermediate shaft 206, the rotary encoder 482a may alternatively be configured to measure the angular position of other components of the delivery apparatus 400 such as the knob 214. In such examples, the scale 483 can be disposed along a circumference of the knob 214.

FIG. 5B shows an axial cross-section of the handle 202 about axis A-A, according to another example. The cross-section shows the rotational sensor 482 embodied as a rotary potentiometer 482b. The rotary potentiometer 482b can comprise a variable resistor whose resistance varies as the rotary potentiometer 482b is rotated about an axis (for example, the central longitudinal axis 220). The rotary potentiometer 482b can be coupled to the intermediate shaft 206 such that the resistance of the rotary potentiometer 482b changes as the intermediate shaft 206 rotates about the central longitudinal axis 220. As illustrated, the rotary potentiometer 482b can be arranged within the handle 202 to measure the angular position of the portion of the intermediate shaft 206 also disposed within the handle 202. However, some examples of the rotary potentiometer 482b can be disposed outside the handle 202, such as at or near the proximal end portion 210 of the intermediate shaft 206 or the knob 214.

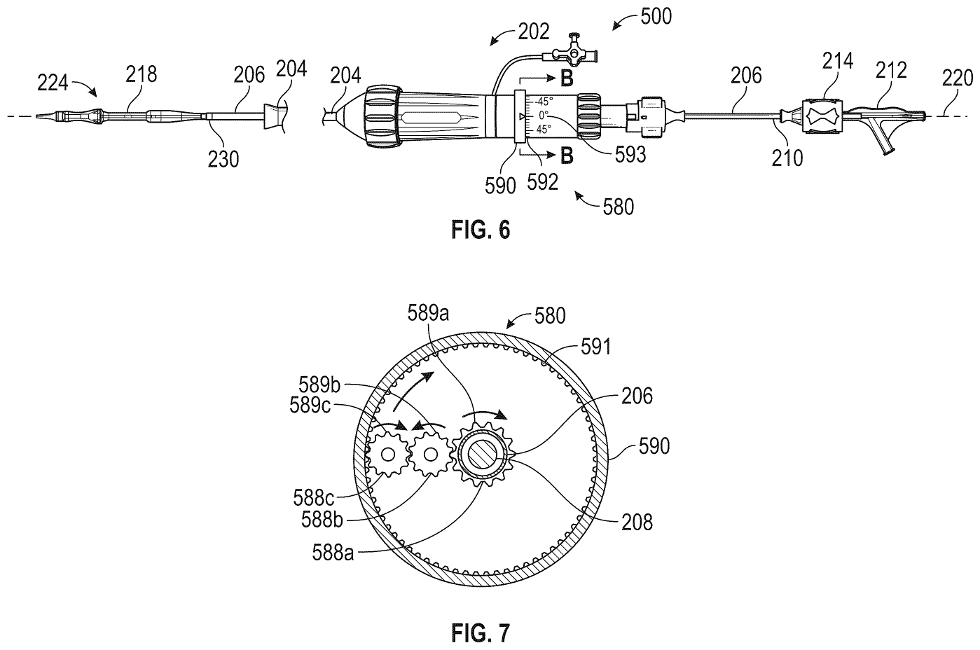

FIG. 6 illustrates a delivery apparatus 500 comprising a mechanical rotation gauge 580 (which is also referred to herein as a “mechanical gauge assembly”), according to an example. The mechanical rotation gauge 580 can be configured to provide a real-time indication of a rotational movement of the prosthetic heart valve about the central longitudinal axis 220. The mechanical rotation gauge 580 can include a gear train comprising one or more gears 588 disposed within the handle 202 and a circumferential ring gear 590 disposed around the handle 202. The mechanical rotation gauge 580 can also include at least one graduated marking 592 circumferentially disposed on the outer surface of the handle 202.

FIG. 7 is an axial cross-section view of the handle 202 of the delivery apparatus 500 about axis B-B. Although the gear train—which includes gears 588 and the ring gear 590—is shown as disposed at or near axis B-B, the various components of the rotation gauge 580 can be disposed anywhere along the axial length of the handle 202 and/or the intermediate shaft 206 (for example, at or near the proximal end portion 210 of the intermediate shaft 206 or at or near the knob 214). Although the illustrated gears 588 comprise a first gear 588a, a second gear 588b, and a third gear 588c, the mechanical rotation gauge 580 can comprise any suitable number of gears 588 arranged in any suitable configuration to mesh the intermediate shaft 206 with the ring gear 590. In some examples, the first gear 588a can circumferentially surround and/or be fixedly coupled to the intermediate shaft 206, such that the first gear 588a can synchronously rotate about the central longitudinal axis 220 in concert with the intermediate shaft 206. In some examples, the first gear 588a can be coupled to the knob 214, such that the first gear 588a rotates at the same rate as the knob 214. The first gear 588a can be in meshed contact with the second gear 588b, which in turn can be in meshed contact with the third gear 588c. The third gear 588c can be in meshed contact with the ring gear 590. In some examples, the ring gear 590 can be concentric with the handle 202 and/or the intermediate shaft 206. In some examples, the ring gear 590 can be configured to rotate about the central longitudinal axis 220. The first gear 588a, the second gear 588b, the third gear 588c, and the ring gear 590 can be meshed such that the gear train has a compound gear ratio between the first gear 588a and the ring gear 590 equal to the transmission ratio of the delivery apparatus 500. For example, the first gear 588a can comprise a first plurality of gear teeth 589a, the second gear 588b can comprise a second plurality of gear teeth 589b, the third gear 588c can comprise a third plurality of gear teeth 589c, and the ring gear can comprise a plurality of ring gear teeth 591. When the ratios of the second plurality of gear teeth 589b to the first plurality of gear teeth 589a, the third plurality of gear teeth 589c to the second plurality of gear teeth 589b, and the plurality of ring gear teeth 591 to the third plurality of gear teeth 589c are multiplied together, the product of the individual gear ratios can result in the compound gear ratio of the gear train, wherein the compound gear ratio is equal to the transmission ratio. As such, the ring gear 590 can rotate about the central longitudinal axis 220 synchronously with or at the same rate as the prosthetic heart valve coupled to the distal end portion of the delivery apparatus 500 when the first gear 588a is rotated with the proximal end portion of the delivery apparatus 500.

In some examples where the distal end portion of the delivery apparatus 500 rotates to a lesser degree than the proximal end portion of the delivery apparatus 500, the transmission ratio and the compound gear ratio can be less than one. However, some examples of the transmission ratio and the compound gear ratio can be equal to or greater than one.

Now referring back to FIG. 6, the at least one graduated marking 592 can represent an angular position of the prosthetic heart valve (or the distal end portion of the delivery apparatus 500) about the central longitudinal axis 220 relative to a reference point 593 (which is also referred to herein as a “reference orientation”) of the handle 202. In the illustrated example, the reference point 593 can comprise the location indicated by the “0°” marking on the handle 202, but it should be understood that the reference point 593 can be any location on the circumference of the handle 202. As the ring gear 590 rotates about the central longitudinal axis 220, the ring gear 590 can rotate relative to the graduated marking 592 which corresponds to the angular position of the prosthetic heart valve relative to the reference point 593 of the handle 202, thereby providing the user with the real-time indication of the rotational movement of the prosthetic heart valve about the central longitudinal axis 220. The graduated marking 592 can be disposed on the handle 202 and adjacent the ring gear 590. However, some examples of the graduated marking 592 can be disposed on any suitable portion of the handle 202 or any other suitable portion of the delivery apparatus 500.

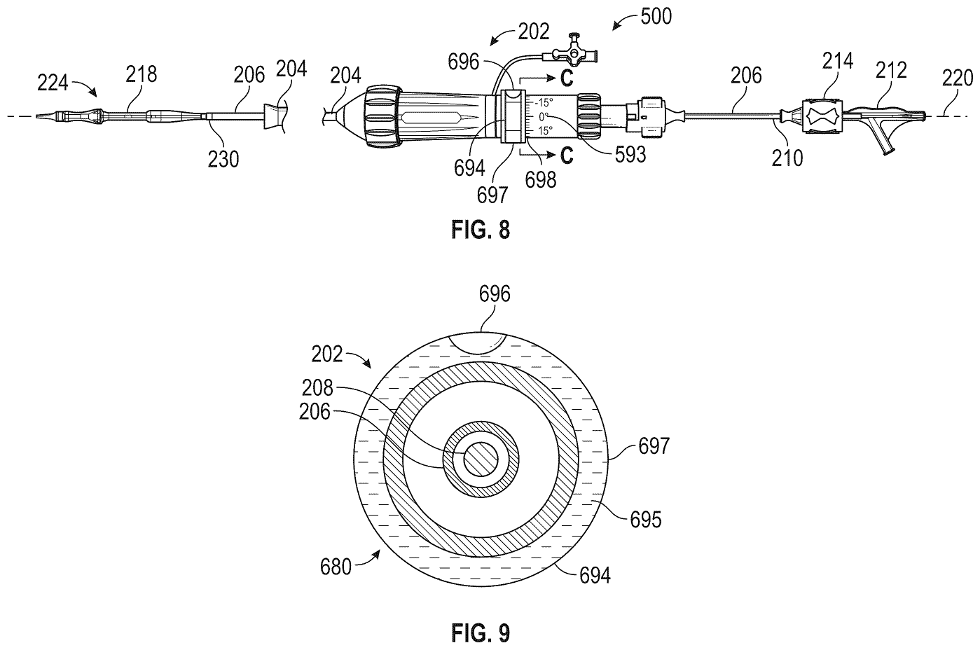

FIG. 8 shows a side view of a delivery apparatus 600 with a spirit level rotation gauge 680, according to an example. The spirit level rotation gauge 680 can be configured to provide a real-time indication of the rotational movement of the prosthetic heart valve about the central longitudinal axis 220. The spirit level rotation gauge 680 can comprise a hollow channel 694 disposed along the circumference of the handle 202, a liquid 695 disposed within the hollow channel 694, a spirit bubble 696 disposed in the liquid 695, a viewport 697 configured to seal the liquid within the channel 694, and at least one graduated marking 698 with which the spirit bubble 696 can align to indicate the rotational movement of the prosthetic heart valve. In some examples where the knob 214 is configured to rotate the proximal end portion 210 of the intermediate shaft 206, the spirit level rotation gauge 680 can instead be disposed on or adjacent the knob 214. In some examples, the spirit level rotation gauge 680 can be disposed on or adjacent the proximal end portion 210 of the intermediate shaft 206.

FIG. 9 is an axial cross-sectional view of the handle 202 of the delivery apparatus 600 about axis C-C. Although the spirit level rotation gauge 680 is shown as disposed at or near axis C-C, the various components of the spirit level rotation gauge 680 can be disposed anywhere along the axial length of the handle 202 and/or the intermediate shaft 206 (for example, at or near the proximal end portion 210 of the intermediate shaft 206 or at or near the knob 214). Although the channel 694 is shown as circumferentially disposed around the outer surface of the handle 202, some examples of the channel 694 may be at least partially embedded within the outer surface or may be flush with the outer surface. The channel 694 can be at least partially filled with the liquid 695, such that the spirit bubble 696 forms within the liquid 695. The liquid 695 can comprise water, an alcohol, an oil, a mineral spirit, or any other suitable fluid. In some examples, the liquid 695 can further comprise a dye or colorant to increase the visibility of the spirit bubble 696. The viewport 697 can comprise a transparent or semi-transparent layer of material configured to seal the liquid 695 within the channel 694, thereby forming an annular vial disposed along the circumference of the handle 202. In some examples, the channel 694 and the viewport 697 may be integrally formed as a single component (for example, the annular vial). The viewport 697 can be configured to allow the user of the delivery apparatus 600 to see the spirit bubble 696 within the spirit level rotation gauge 680.

Now referring back to FIG. 8, the at least one graduated marking 698 can be configured to indicate the angular position of the prosthetic heart valve about the central longitudinal axis 220 relative to the reference point 593 when the spirit bubble 696 aligns with the graduated marking 698. The graduated marking 698 can be disposed a first angular displacement (which is also referred to herein as an “angular distance” or an “angle”) from the reference point 593 on the handle 202. When the graduated marking 698 comes into alignment with the spirit bubble 696, the graduated marking 698 can be indicative of the prosthetic heart valve being disposed a second angular displacement from the reference point 593 on the handle 202, wherein the second angular displacement is offset from the first angular displacement by the transmission ratio. In some examples where the offset is multiplicative, the second angular displacement can be the product or quotient of the first angular displacement and the transmission ratio. In some examples where the offset is additive, the second angular displacement can be the sum or difference of the first angular displacement and the transmission ratio. However, the offset from the first angular displacement by the transmission ratio can be any other mathematical relationship (exponential, logarithmic, non-linear, etc.). As the handle 202 is rotated about the central longitudinal axis 220, the spirit bubble 696 will naturally move within or migrate along the channel 694 towards the top of the spirit level rotation gauge 680 (for example, a top-most portion of the channel 694) to align with the graduated marking 698, thereby indicating the angular position of the prosthetic heart valve relative to the reference point 593 point on the handle 202.

Although the illustrated graduated marking 698 is disposed on the outer surface of the handle and adjacent the channel 694, the graduated marking 698 can alternatively be disposed on an outer surface of the viewport 697.

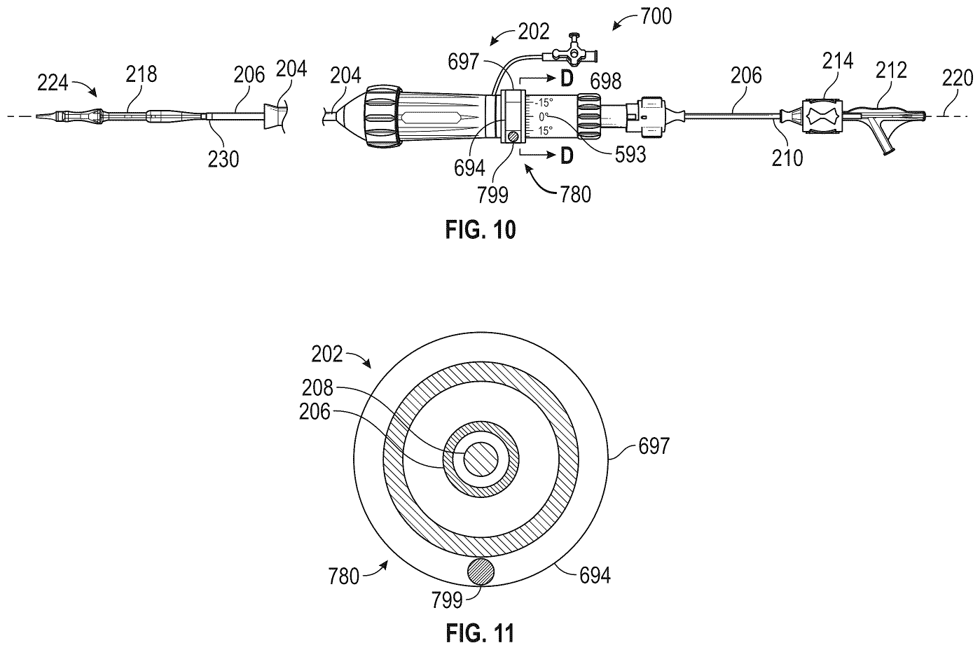

FIG. 10 shows a side view of a delivery apparatus 700 with a ball bearing rotation gauge 780, according to an example. The ball bearing rotation gauge 780 can be configured to provide a real-time indication of a rotational movement of the prosthetic heart valve about the central longitudinal axis 220. The ball bearing rotation gauge 780 can comprise similar features as the spirit level rotation gauge 680 illustrated in FIGS. 8-9. However, one exemplary difference between the ball bearing rotation gauge 780 and the spirit level rotation gauge 680 is that the ball bearing rotation gauge 780 can comprise a ball bearing 799 disposed in the channel 694 instead of the spirit bubble 696 and the liquid 695.

FIG. 11 is an axial cross-sectional view of the handle 202 of the delivery apparatus 700 about axis D-D. Although the ball bearing rotation gauge 780 is shown as disposed at or near axis D-D, the various components of the ball bearing rotation gauge 780 can be disposed anywhere along the axial length of the handle 202 and/or the intermediate shaft 206 (for example, at or near the proximal end portion 210 of the intermediate shaft 206 or at or near the knob 214). The ball bearing 799 can comprise a weighted ball is configured to move within or migrate along the channel 694 to a bottom-most portion of the channel 694. When the ball bearing 799 reaches the bottom-most portion of the channel 694, the ball bearing 799 can align with the graduated marking 698, thereby indicating the angular position of the prosthetic heart valve relative to the reference point 593 point on the handle 202.

### Computing System

FIG. 12 depicts an example of a suitable computing system 800 in which the described electronic rotation gauges 380, 480 can be implemented. The computing system 800 is not intended to suggest any limitation as to scope of use or functionality of the present disclosure, as the innovations can be implemented in diverse computing systems.

With reference to FIG. 12, the computing system 800 can include one or more processing units 810, 815 and memory 820, 825. In FIG. 12, this basic configuration 830 is included within a dashed line. The processing units 810, 815 (which can be similar to controller 384) can execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units can execute computer-executable instructions to increase processing power. For example, FIG. 12 shows a central processing unit 810 as well as a graphics processing unit or co-processing unit 815. The tangible memory 820, 825 can be volatile memory (such as registers, cache, RAM), non-volatile memory (such as ROM, EEPROM, flash memory, etc.), or some combination of the two, accessible by the processing unit(s) 810, 815. The memory 820, 825 can store software implementing one or more innovations described herein, in the form of computer-executable instructions suitable for execution by the processing unit(s) 810, 815.

The computing system 800 can have additional features. For example, the computing system 800 can include storage 840, one or more input devices 850 (such as the IMU 382, the rotary encoder 482a, and the rotary potentiometer 482b described above), one or more output devices 860 (such as the electronic display 386 described above), and one or more communication connections 870, including input devices, output devices, and communication connections for interacting with a user. An interconnection mechanism such as a bus, controller, or network can interconnect the components of the computing system 800. Typically, operating system software can provide an operating environment for other software executing in the computing system 800, and coordinates activities of the components of the computing system 800.

The tangible storage 840 can be removable or non-removable, and includes magnetic disks, magnetic tapes or cassettes, CD-ROMs, DVDs, or any other medium which can be used to store information in a non-transitory way and which can be accessed within the computing system 800. The storage 840 can store instructions for the software implementing one or more innovations described herein.

The input device(s) 850 can be an input device such as a keyboard, mouse, pen, or trackball, a voice input device, a scanning device, touch device (such as a touchpad, a display, or the like) or another device that provides input to the computing system 800. The output device(s) 860 can be a display, printer, speaker, CD-writer, or another device that provides output from the computing system 800. For example, the input device(s) 850 can include the one or more sensors (such as the IMU 382, the rotary encoder 482a, and/or the rotary potentiometer 482b) described above. Some examples of the output device(s) 860 can include the electronic display 386 described above.

The communication connection(s) 870 can enable communication over a communication medium to another computing entity. The communication medium can convey information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier. In one specific example, the communication connection(s) 870 can include a communication unit (e.g., a Bluetooth module) which is configured to wirelessly communicate with a mobile computing device.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various examples. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer and should not be confused with acts performed by a human being. The actual computer operations corresponding to these terms vary depending on implementation.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising:
a shaft comprising a proximal end portion, a distal end portion, and a central longitudinal axis extending from the proximal end portion to the distal end portion, wherein the distal end portion of the shaft is configured to be coupled to a prosthetic medical device;
a handle coupled to the proximal end portion of the shaft; and
a rotation gauge coupled to the proximal end portion of the shaft, wherein the rotation gauge is configured to provide a real-time indication of a rotational movement of the prosthetic medical device about the central longitudinal axis based on a rotational movement of the proximal end portion of the shaft and a transmission ratio of the shaft.

Example 2. The delivery apparatus of any example herein, particularly Example 1, wherein the transmission ratio is a ratio of the rotational movement of the distal end portion of the shaft to the rotational movement of the proximal end portion of the shaft.

Example 3. The delivery apparatus of any example herein, particularly any one of Examples 1-2, wherein the rotational movement of the proximal end portion of the shaft results in the rotational movement of the prosthetic medical device coupled to the distal end portion of the shaft.

Example 4. The delivery apparatus of any example herein, particularly any one of Examples 1-3, wherein the transmission ratio is less than one.

Example 5. A delivery apparatus comprising:
a shaft extending along a central longitudinal axis, the shaft comprising a proximal end portion and a distal end portion;
a handle coupled to the proximal end portion of the shaft; and
a rotation gauge coupled to the handle, wherein:
   the rotation gauge is configured to provide an indication of a rotational movement of the distal end portion of the shaft about the central longitudinal axis, and
   the indication is based on a rotational movement of the proximal end portion of the shaft about to the central longitudinal axis and a transmission ratio between the distal end portion of the shaft and the proximal end portion of the shaft.

Example 6. The delivery apparatus of any example herein, particularly Example 5, wherein the rotation gauge comprises a rotational sensor configured to measure the rotational movement of the proximal end portion of the shaft.

Example 7. The delivery apparatus of any example herein, particularly Example 6, wherein the rotational sensor comprises one of a rotary potentiometer and a rotary encoder.

Example 8. The delivery apparatus of any example herein, particularly any one of Examples 5-7, wherein the rotation gauge comprises a processor configured to determine the indication of the rotational movement of the distal end portion of the shaft by multiplying the rotational movement of the proximal end portion of the shaft about the central longitudinal axis by the transmission ratio.

Example 9. The delivery apparatus of any example herein, particularly any one of Examples 5-8, wherein the rotation gauge further comprises an electronic display configured to display the indication of the rotational movement of the distal end portion of the shaft.

Example 10. The delivery apparatus of any example herein, particularly Example 9, wherein the electronic display is disposed on the handle.

Example 11. The delivery apparatus of any example herein, particularly Example 5, wherein the rotation gauge includes a gear train comprising a gear fixedly coupled to the shaft and a circumferential ring gear disposed around the handle.

Example 12. The delivery apparatus of any example herein, particularly Example 11, wherein the gear train defines a compound gear ratio, and wherein the compound gear ratio is equal to the transmission ratio such that the circumferential ring gear is configured to rotate about the central longitudinal axis at the same rate as the distal end portion of the shaft.

Example 13. The delivery apparatus of any example herein, particularly any one of Examples 11-12, wherein the rotation gauge further comprises a graduated marking disposed on an outer surface of the handle, wherein the graduated marking corresponds to an angular position of the distal end portion of the shaft relative to a reference point of the handle.

Example 14. The delivery apparatus of any example herein, particularly Example 13, wherein the rotation gauge is configured to provide the indication of the rotational movement of the distal end portion of the shaft by rotating the circumferential ring gear about the central longitudinal axis relative to the graduated marking.

Example 15. The delivery apparatus of any example herein, particularly any one of Examples 5-14, wherein the indication of the rotational movement of the distal end portion of the shaft about the central longitudinal axis is relative to a reference orientation of the handle.

Example 16. A delivery apparatus for implanting a prosthetic medical device, the delivery apparatus comprising:
a shaft extending along a central longitudinal axis, the shaft comprising a proximal end portion and a distal end portion;
a valve mounting portion coupled to the distal end portion of the shaft, wherein the valve mounting portion is configured to receive the prosthetic medical device;
a handle coupled to the proximal end portion of the shaft; and
a rotation gauge disposed on the handle, wherein the rotation gauge is configured to provide an indication of a degree of rotational movement of the valve mounting portion about the central longitudinal axis based on a degree of rotational movement of the handle about the central longitudinal axis and a transmission ratio between the distal end portion of the shaft and the handle.

Example 17. The delivery apparatus of any example herein, particularly Example 16, wherein the rotation gauge comprises a gyroscope fixedly coupled to the handle, wherein the gyroscope is configured to measure the degree of rotational movement of the handle about the central longitudinal axis.

Example 18. The delivery apparatus of any example herein, particularly Example 16, wherein the rotation gauge comprises an annular vial disposed along a circumference of the handle and a spirit bubble disposed in the annular vial, wherein rotational movement of the handle about the central longitudinal axis results in the spirit bubble moving within the annular vial relative to a reference point of the handle to provide the indication of the degree of rotational movement of the valve mounting portion about the central longitudinal axis.

Example 19. The delivery apparatus of any example herein, particularly Example 16, wherein the rotation gauge comprises a channel disposed along a circumference of the handle and a ball bearing disposed within the channel, wherein the ball bearing is configured to migrate along the channel relative to a reference point of the handle as the handle is rotated about the central longitudinal axis.

Example 20. A method of providing an indication of a rotational movement of a prosthetic heart valve coupled to a distal end portion of a delivery apparatus, the method comprising:
measuring an angular position of a proximal end portion of the delivery apparatus relative to a central longitudinal axis of the delivery apparatus;
determining an angular position of the prosthetic heart valve relative to the central longitudinal axis by multiplying the angular position of the proximal end portion of the delivery apparatus by a transmission ratio, wherein the transmission ratio is a ratio of a rotational movement of the distal end portion of the delivery apparatus to a rotational movement of the proximal end portion of the delivery apparatus; and displaying the angular position of the prosthetic heart valve.

Example 21. The delivery apparatus of any example herein, particularly any one of Examples 1-20, wherein the delivery apparatus is sterilized.

The features described herein with regard to any example can be combined with other features described in any one or more of the other examples, unless otherwise stated. For example, any one or more of the features of hub assembly support can be combined with any one or more features of another hub assembly support. As another example, any one or more features of one docking device delivery apparatus can be combined with any one or more features of another docking device delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a shaft comprising a proximal end portion, a distal end portion, and a central longitudinal axis extending from the proximal end portion to the distal end portion, wherein the distal end portion of the shaft is configured to be coupled to a prosthetic medical device;
a handle coupled to the proximal end portion of the shaft; and
a rotation gauge coupled to the proximal end portion of the shaft, wherein the rotation gauge is configured to provide a real-time indication of a rotational movement of the prosthetic medical device about the central longitudinal axis based on a rotational movement of the proximal end portion of the shaft and a transmission ratio of the shaft.

2. The delivery apparatus of claim 1, wherein the transmission ratio is a ratio of the rotational movement of the distal end portion of the shaft to the rotational movement of the proximal end portion of the shaft.

3. The delivery apparatus of claim 1, wherein the rotational movement of the proximal end portion of the shaft results in the rotational movement of the prosthetic medical device coupled to the distal end portion of the shaft.

4. The delivery apparatus of claim 1, wherein the transmission ratio is less than one.

5. A delivery apparatus comprising:
a shaft extending along a central longitudinal axis, the shaft comprising a proximal end portion and a distal end portion;
a handle coupled to the proximal end portion of the shaft; and
a rotation gauge coupled to the handle, wherein:
the rotation gauge is configured to provide an indication of a rotational movement of the distal end portion of the shaft about the central longitudinal axis, and
the indication is based on a rotational movement of the proximal end portion of the shaft about to the central longitudinal axis and a transmission ratio between the distal end portion of the shaft and the proximal end portion of the shaft.

6. The delivery apparatus of claim 5, wherein the rotation gauge comprises a rotational sensor configured to measure the rotational movement of the proximal end portion of the shaft.

7. The delivery apparatus of claim 6, wherein the rotational sensor comprises one of a rotary potentiometer and a rotary encoder.

8. The delivery apparatus of claim 5, wherein the rotation gauge comprises a processor configured to determine the indication of the rotational movement of the distal end portion of the shaft by multiplying the rotational movement of the proximal end portion of the shaft about the central longitudinal axis by the transmission ratio.

9. The delivery apparatus of claim 5, wherein the rotation gauge further comprises an electronic display configured to display the indication of the rotational movement of the distal end portion of the shaft.

10. The delivery apparatus of claim 9, wherein the electronic display is disposed on the handle.

11. The delivery apparatus of claim 5, wherein the rotation gauge includes a gear train comprising a gear fixedly coupled to the shaft and a circumferential ring gear disposed around the handle.

12. The delivery apparatus of claim 11, wherein the gear train defines a compound gear ratio, and wherein the compound gear ratio is equal to the transmission ratio such that the circumferential ring gear is configured to rotate about the central longitudinal axis at the same rate as the distal end portion of the shaft.

13. The delivery apparatus of claim 11, wherein the rotation gauge further comprises a graduated marking disposed on an outer surface of the handle, wherein the graduated marking corresponds to an angular position of the distal end portion of the shaft relative to a reference point of the handle.

14. The delivery apparatus of claim 13, wherein the rotation gauge is configured to provide the indication of the rotational movement of the distal end portion of the shaft by rotating the circumferential ring gear about the central longitudinal axis relative to the graduated marking.

15. The delivery apparatus of claim 5, wherein the indication of the rotational movement of the distal end portion of the shaft about the central longitudinal axis is relative to a reference orientation of the handle.

16. A delivery apparatus for implanting a prosthetic medical device, the delivery apparatus comprising:
A shaft extending along a central longitudinal axis, the shaft comprising a proximal end portion and a distal end portion;
a valve mounting portion coupled to the distal end portion of the shaft, wherein the valve mounting portion is configured to receive the prosthetic medical device;
a handle coupled to the proximal end portion of the shaft; and
a rotation gauge disposed on the handle, wherein the rotation gauge is configured to provide an indication of a degree of rotational movement of the valve mounting portion about the central longitudinal axis based on a degree of rotational movement of the handle about the central longitudinal axis and a transmission ratio between the distal end portion of the shaft and the handle.

17. The delivery apparatus of claim 16, wherein the rotation gauge comprises a gyroscope fixedly coupled to the handle, wherein the gyroscope is configured to measure the degree of rotational movement of the handle about the central longitudinal axis.

18. The delivery apparatus of claim 16, wherein the rotation gauge comprises an annular vial disposed along a circumference of the handle and a spirit bubble disposed in the annular vial, wherein rotational movement of the handle about the central longitudinal axis results in the spirit bubble moving within the annular vial relative to a reference point of the handle to provide the indication of the degree of rotational movement of the valve mounting portion about the central longitudinal axis.

19. The delivery apparatus of claim 16, wherein the rotation gauge comprises a channel disposed along a circumference of the handle and a ball bearing disposed within the channel, wherein the ball bearing is configured to migrate along the channel relative to a reference point of the handle as the handle is rotated about the central longitudinal axis.
